(19) United States
(12) Patent Application Publication
(10) Pub. No.: US 2025/0172484 A1
(43) Pub. Date: May 29, 2025

(54) Classification Model Generation Method, Particle Classification Method, and Recording Medium

(71) Applicants: ThinkCyte K.K., Tokyo (JP); The University of Tokyo, Tokyo (JP)

(72) Inventors: Hiroaki Adachi, Tokyo (JP); Issei Sato, Tokyo (JP); Sadao Ota, Tokyo (JP)

(21) Appl. No.: 18/688,917

(22) PCT Filed: Aug. 29, 2022
(86) PCT No.: PCT/JP2022/032308

§ 371 Date: Nov. 6, 2024

(30) Foreign Application Priority Data
Sep. 17, 2021 (JP) 2021-152461

(51) Int. Cl. G01N 15/1429

(52) U.S. Cl. CPC G01N 15/1429

(57) ABSTRACT

A classification model that outputs identification information indicating whether or not a particle has specific morphological characteristics when waveform data is input is generated by training using training data including first waveform data that is obtained by irradiating light to particles contained in a first sample and having specific morphological characteristics and indicates morphological characteristics of the particles, information indicating that the first waveform data has been obtained from particles contained in the first sample, second waveform data indicating morphological characteristics of unspecified particles contained in a second sample, information indicating that the second waveform data has been obtained from particles contained in the second sample, and a positive rate that is the proportion of particles having the specific morphological characteristics.

### REFERENCE TO RELATED APPLICATIONS

This application is the national phase under 35 U. S. C. § 371 of PCT International Application No. PCT/JP2022/032308 which has an International filing date of Aug. 29, 2022 and designated the United States of America.

### BACKGROUND ART

Conventionally, a flow cytometry method has been used as a method for examining individual cells. The flow cytometry method is a cell analysis method for acquiring information regarding cells irradiated with light as a captured image or the like by making cells dispersed in a fluid pass through a channel, irradiating light to each cell moving through the channel, and measuring scattered light or fluorescence from the cells irradiated with light. By using the flow cytometry method, a large number of cells can be analyzed one by one at high speed.

In addition, a ghost cytometry method (hereinafter, referred to as GC method) has been developed in flow cytometers in which cells moving through a channel are irradiated with special structured illumination light, waveform data of the optical signal including compressed morphological information of cells is acquired from the cells, and the cells are classified based on the waveform data. An example of the GC method is disclosed in International Publication No. 2017/073737. In the GC method, a classification model for classifying cells is created in advance by machine learning from waveform data of a training sample, and cells contained in a test sample are classified using the classification model. Thus, in flow cytometers using the GC method, a classification model is created by machine learning using one-dimensional waveform data including compressed morphological characteristics of cells as training data as it is, and cells are classified using the created classification model. This enables faster processing.

### SUMMARY

One of the uses of the flow cytometer using the GC method is to classify cells in order to identify cells having specific morphological characteristics from other cells based on cell morphology. For example, as an example, by modifying the genes of a cell using a gene editing technique and acquiring a cell exhibiting a specific cell phenotype, a genetically modified site in the cell may be identified. Another example is cell phenotype screening for selecting a test substance that changes the phenotype of a cell to a cell having specific morphological characteristics. In such cases, there is a need to identify cells exhibiting a predetermined phenotype, among cells that have been genetically modified by using a gene editing technique or treated by contact with a test substance, based on their morphological characteristics. Here, cells to be identified are referred to as positive cells, and the other cells are referred to as negative cells. The morphological characteristics of positive cells are one type. In contrast, negative cells have different morphological characteristics from positive cells, and their morphological characteristics vary. Supervised machine learning used in the conventional GC method requires both waveform data of positive cells and waveform data of negative cells as training data in order to create a classification model.

However, in the cases described above, it may not be possible to prepare waveform data reflecting the diversity of negative cells. For example, it is possible to create positive cells by modifying known genes known to be associated with the desired cell phenotype or by contacting the cells with a chemical known to convert cells to a phenotype having specific morphological characteristics and to obtain waveform data of the positive cells. On the other hand, samples containing negative cells can be obtained, for example, by modifying a known gene whose association with the desired cell phenotype is unknown or by contacting the cells with a chemical whose effect on converting cells to a phenotype having specific morphological characteristics has not been confirmed. Such a sample containing negative cells is a mixture of various cells having different morphological characteristics. If such a mixture of unspecified cells can be created as a training sample, it is possible to perform training using waveform data of negative cells. However, it is almost impractical to create samples containing various cells having different morphological characteristics, which are obtained by genetic modification or by contacting the cells with a chemical, and prepare training data reflecting the morphological characteristics of the mixture of unspecified cells. In addition, there is a possibility that cells having morphological characteristics similar to those of positive cells will be included. Therefore, it has been difficult to perform training that reflects all the diversity of morphological characteristics of cell populations to be classified.

The present disclosure has been made in view of the above circumstances, and it is an object to provide a classification model generation method, a particle classification method, a computer program, and an information processing device for classifying particles by using waveform data indicating specific morphological characteristics and waveform data indicating unspecified morphological characteristics as training data.

A classification model generation method according to an aspect of the present disclosure, is characterized by comprising: acquiring first waveform data, which is obtained by irradiating light to particles contained in a first sample formed of particles having specific morphological characteristics and indicates morphological characteristics of the particles, and second waveform data indicating morphological characteristics of particles contained in a second sample formed of a plurality of unspecified particles; and generating a classification model, which outputs identification information indicating whether or not a particle has the specific morphological characteristics when waveform data indicating morphological characteristics of the particle is input, by training using training data including the first waveform data, information indicating that the first waveform data has been obtained from particles contained in the first sample, the second waveform data, information indicating that the second waveform data has been obtained from particles contained in the second sample, and a positive rate that is a proportion of particles having the specific morphological characteristics among all particles contained in the first sample and the second sample.

In the classification model generation method according to an aspect of the present disclosure, it is characterized in that the positive rate is a value obtained by measuring a proportion of particles having the specific morphological characteristics contained in a mixed sample obtained by mixing the first sample and the second sample together or a value obtained by calculating a proportion of particles having the specific morphological characteristics among all particles contained in the first sample and the second sample.

In the classification model generation method according to an aspect of the present disclosure, it is characterized in that the waveform data is waveform data indicating a temporal change in an intensity of light emitted from a particle irradiated with light by a structured illumination or waveform data indicating a temporal change in an intensity of light detected by structuring light from a particle irradiated with light.

In the classification model generation method according to an aspect of the present disclosure, it is characterized in that by using a part of a mixed sample obtained by mixing the first sample and the second sample together as a training sample, the first waveform data and the second waveform data obtained from particles contained in the training sample are acquired as waveform data contained in the training data, and the classification model is trained to output identification information indicating whether or not a particle contained in the mixed sample has the specific morphological characteristics when waveform data obtained from the particle is input.

A particle classification method according to an aspect of the present disclosure, is characterized by comprising: inputting waveform data indicating morphological characteristics of a particle to a classification model that outputs identification information indicating whether or not the particle has specific morphological characteristics when waveform data, which is obtained by irradiating light to the particle and indicates morphological characteristics of the particle, is input; and determining whether or not the particle has the specific morphological characteristics based on the identification information output from the classification model, wherein the classification model is trained by using training data including first waveform data indicating morphological characteristics of particles contained in a first sample formed of particles having the specific morphological characteristics, information indicating that the first waveform data has been obtained from particles contained in the first sample, second waveform data indicating morphological characteristics of particles contained in a second sample formed of a plurality of unspecified particles, information indicating that the second waveform data has been obtained from particles contained in the second sample, and a positive rate that is a proportion of particles having the specific morphological characteristics among all particles contained in the first sample and the second sample.

In the particle classification method according to an aspect of the present disclosure, it is characterized in that waveform data indicating morphological characteristics of particles contained in a mixed sample obtained by mixing the first sample and the second sample together is acquired, waveform data obtained from the particles contained in the mixed sample is input to the classification model, and whether or not each particle contained in the mixed sample has the specific morphological characteristics is determined based on identification information output from the classification model.

In the particle classification method according to an aspect of the present disclosure, it is characterized in that particles contained in the first sample are stained, particles contained in the second sample are not stained, and particles that are not stained and have the specific morphological characteristics are identified based on presence or absence of staining of each particle determined to be a particle having the specific morphological characteristics.

A computer program according to an aspect of the present disclosure, is characterized by causing a computer to execute processing of: acquiring first waveform data, which is obtained by irradiating light to particles contained in a first sample formed of particles having specific morphological characteristics and indicates morphological characteristics of the particles, and second waveform data indicating morphological characteristics of particles contained in a second sample formed of a plurality of unspecified particles; and generating a classification model, which outputs identification information indicating whether or not a particle has the specific morphological characteristics when waveform data indicating morphological characteristics of the particle is input, by training using training data including the first waveform data, information indicating that the first waveform data has been obtained from particles contained in the first sample, the second waveform data, information indicating that the second waveform data has been obtained from particles contained in the second sample, and a positive rate that is a proportion of particles having the specific morphological characteristics among all particles contained in the first sample and the second sample.

An information processing device according to an aspect of the present disclosure, is characterized by comprising: a data acquisition unit that acquires first waveform data, which is obtained by irradiating light to particles contained in a first sample formed of particles having specific morphological characteristics and indicates morphological characteristics of the particles, and second waveform data indicating morphological characteristics of particles contained in a second sample formed of a plurality of unspecified particles; and a classification model generation unit that generates a classification model, which outputs identification information indicating whether or not a particle has the specific morphological characteristics when waveform data indicating morphological characteristics of the particle is input, by training using training data including the first waveform data, information indicating that the first waveform data has been obtained from particles contained in the first sample, the second waveform data, information indicating that the second waveform data has been obtained from particles contained in the second sample, and a positive rate that is a proportion of particles having the specific morphological characteristics among all particles contained in the first sample and the second sample.

A computer program according to an aspect of the present disclosure, is characterized by causing a computer to execute processing of: inputting waveform data indicating morphological characteristics of a particle to a classification model that outputs identification information indicating whether or not the particle has specific morphological characteristics when waveform data, which is obtained by irradiating light to the particle and indicates morphological characteristics of the particle, is input; and determining whether or not the particle has the specific morphological characteristics based on the identification information output from the classification model, wherein the classification model is trained by using training data including first waveform data indicating morphological characteristics of particles contained in a first sample formed of particles having the specific morphological characteristics, information indicating that the first waveform data has been obtained from particles contained in the first sample, second waveform data indicating morphological characteristics of particles contained in a second sample formed of a plurality of unspecified particles, information indicating that the second waveform data has been obtained from particles contained in the second sample, and a positive rate that is a proportion of particles having the specific morphological characteristics among all particles contained in the first sample and the second sample.

An information processing device according to an aspect of the present disclosure, is characterized by comprising: a data input unit that inputs waveform data indicating morphological characteristics of a particle to a classification model that outputs identification information indicating whether or not the particle has specific morphological characteristics when waveform data, which is obtained by irradiating light to the particle and indicates morphological characteristics of the particle, is input; and a determination unit that determines whether or not the particle has the specific morphological characteristics based on the identification information output from the classification model, wherein the classification model is trained by using training data including first waveform data indicating morphological characteristics of particles contained in a first sample formed of particles having the specific morphological characteristics, information indicating that the first waveform data has been obtained from particles contained in the first sample, second waveform data indicating morphological characteristics of particles contained in a second sample formed of a plurality of unspecified particles, information indicating that the second waveform data has been obtained from particles contained in the second sample, and a positive rate that is a proportion of particles having the specific morphological characteristics among all particles contained in the first sample and the second sample.

According to one aspect of the present disclosure, the classification model is trained by using the training data including the first waveform data obtained from particles contained in the first sample formed of particles having specific morphological characteristics, the second waveform data obtained from particles contained in the second sample formed of unspecified particles, and the positive rate that is the proportion of particles having the specific morphological characteristics. The waveform data indicates the morphological characteristics of the particles. When the waveform data is input, the classification model outputs the identification information indicating whether or not the particle has the specific morphological characteristics. The classification model can be trained by using training data including the second waveform data obtained from unspecified particles.

According to one aspect of the present disclosure, the positive rate is a value indicating the proportion of particles having specific morphological characteristics contained in a mixed sample obtained by mixing the first sample and the second sample. For example, the positive rate is obtained by measuring the proportion of particles having the specific morphological characteristics that are actually contained in the mixed sample. Alternatively, the positive rate can be obtained by calculating the proportion of particles having the specific morphological characteristics among all particles contained in the first sample and the second sample. In addition, the second sample contains a variety of particles, and the number of particles having specific morphological characteristics in the second sample may be very small. In this case, the ratio of the number of particles contained in the first sample to the total number of particles contained in the first and second samples is approximately equal to the positive rate, and this value can be used as the positive rate during training.

According to one aspect of the present disclosure, the waveform data is waveform data indicating a temporal change in the intensity of light emitted from a particle irradiated with light by a structured illumination or waveform data indicating a temporal change in the intensity of light detected by structuring light from a particle irradiated with light. The waveform data is similar to that used in the GC method, and indicates the morphological characteristics of the particle.

According to one aspect of the present disclosure, by using a part of the mixed sample obtained by mixing the first sample and the second sample together as a training sample, the first waveform data and the second waveform data obtained from particles contained in the training sample are used as training data. The classification model is trained to output identification information indicating whether or not the particle contained in the mixed sample has specific morphological characteristics. The classification model can be trained by using a part of the mixed sample, and particles contained in the remaining mixed sample can be classified by using the classification model.

According to one aspect of the present disclosure, waveform data is input to the classification model according to the present disclosure, and it is determined whether or not the particle has specific morphological characteristics based on the identification information output from the classification model. Even if the waveform data of particles having morphological characteristics other than the specific morphological characteristics cannot be used as training data, it is possible to classify particles using the GC method.

According to one aspect of the present disclosure, waveform data obtained from particles contained in the mixed sample obtained by mixing the first sample and the second sample together is input to the classification model to classify the particles. For particles contained in the remainder of the mixed sample used to train the classification model, classification as to whether or not the particle has the specific morphological characteristics can be performed by using the classification model.

According to one aspect of the present disclosure, particles contained in the first sample are stained, and particles contained in the second sample are not stained. By identifying particles, which are not stained and have the specific morphological characteristics, from the mixed sample based on the presence or absence of staining, it is possible to easily identify particles having the specific morphological characteristics contained in the second sample.

One aspect of the present disclosure has excellent effects, such as being able to generate a classification model for determining whether or not each particle has specific morphological characteristics even if waveform data regarding particles having morphological characteristics other than the specific morphological characteristics cannot be used as training data.

The above and further objects and features will more fully be apparent from the following detailed description with accompanying drawings.

### BRIEF DESCRIPTION OF DRAWINGS

FIG. 1 is a conceptual diagram showing the rough procedure of a cell classification method.

FIG. 2 is a block diagram showing a configuration example of a classification apparatus according to a first embodiment for performing training and cell classification.

FIG. 3 is a graph showing an example of waveform data.

FIG. 4 is a block diagram showing an example of the internal configuration of an information processing device.

FIG. 5 is a conceptual diagram showing the functions of a classification model.

FIG. 6 is a flowchart showing an example of a procedure of processing for training a classification model.

FIG. 7 is a flowchart showing an example of the procedure of processing performed by an information processing device in order to classify cells.

FIG. 8 is a block diagram showing a configuration example of a classification apparatus according to a second embodiment.

### DESCRIPTION

Hereinafter, the present disclosure will be specifically described with reference to the diagrams showing embodiments thereof.

### First Embodiment

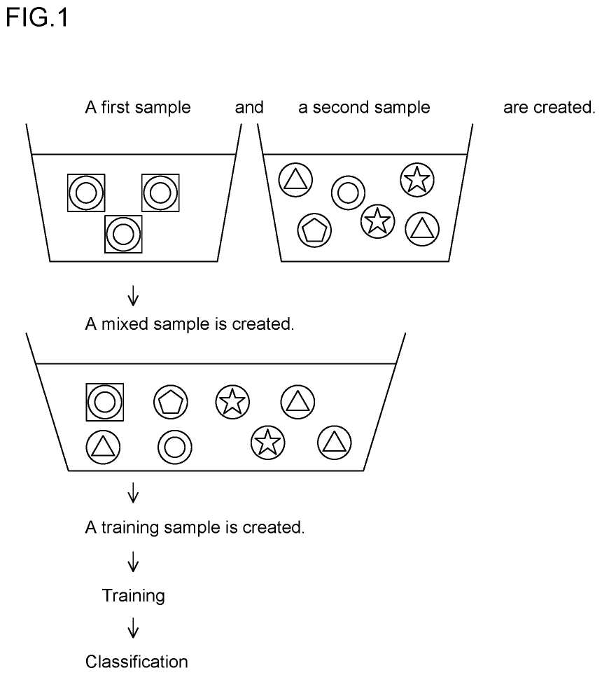

FIG. 1 is a conceptual diagram showing the rough procedure of a cell classification method. Cells are an example of particles to be classified. In the present embodiment, cells are classified in order to identify cells having specific morphological characteristics among cells having various morphological characteristics. The cells may be any cells, such as human cells, animal cells, or microbial cells. In the following description, identifying cells in which nuclear translocation of NF-κB (nuclear factor-kappa B) by LPS (lipopolysaccharide) stimulation is inhibited, that is, cells in which NF-κB remains in the cytoplasm without translocating to the nucleus even when LPS is applied, among cells whose genes have been modified in various ways by using a gene editing technique, is shown as a main example. In this description, cells in which nuclear translocation of NP-κB is inhibited are examples of cells having specific morphological characteristics. Hereinafter, cells having specific morphological characteristics will be referred to as positive cells, and cells having morphological characteristics other than the specific morphological characteristics will be referred to as negative cells. In the cell classification method, first, a first sample and a second sample are prepared. The cells contained in the first sample are only positive cells having specific morphological characteristics. The second sample is a sample containing a plurality of cells having unspecified morphological characteristics. As a method for creating a plurality of cells that are contained in the second sample and have unspecified morphological characteristics, for example, known gene editing techniques can be used to modify the genes of a cell in various ways (cutting of a gene, excision of a part of a gene, insertion of a new gene, and the like). As gene editing techniques, CRISPR systems such as CRISPR (Clustered Regularly Interspaced Short Palindromic Repeats)-Cas9 (Crispr Associated protein 9), ZFN (Zinc-Finger Nuclease), and TALEN (Transcription Activator-Like Effector Nuclease) can be used. Genetically modified cells can express different morphological characteristics due to genetic modification. In addition, another example of the method for creating a plurality of cells having unspecified morphological characteristics is a method in which cells are brought into contact with different test substances and the contact causes the cells to express specific morphological characteristics. In this case, there are two possible cases of a case where the morphological characteristics are expressed by the direct effect of the test substance on cells and a case where the morphological characteristics are expressed by the inhibitory effect of the test substance on a certain agent (a chemical, a physiologically active substance, or the like).

By collecting a plurality of cells exhibiting variously different morphological characteristics through the treatment described above, a second sample is created. The second sample can be created by treating a plurality of cells separately and mixing the plurality of created cells together. The second sample can also be created by simultaneously and randomly performing gene editing operations on a plurality of cells or by simultaneously and randomly contacting the plurality of cells with a test substance rather than creating a plurality of cells separately and then mixing the plurality of cells. Alternatively, the second sample may be created by treating a plurality of cells separately up to an intermediate step, collecting the cells, and then performing the subsequent steps simultaneously on the plurality of cells. The second sample is a sample containing cells having unspecified morphological characteristics. The second sample contains positive cells and negative cells. In the second sample, the number of positive cells is often, but not necessarily, less than the number of negative cells. The number of positive cells contained in the second sample may be small. When creating cells that express morphological characteristics through gene editing or contact between cells and a test substance, the second sample may contain no positive cells or the number of positive cells may be extremely small.

For example, the second sample can be created by creating cells that have undergone variously different genetic modifications with the gene editing technique and injecting LPS, which causes nuclear translocation of NF-κB, into a mixed sample of a plurality of cells that have undergone different gene editing. By collecting cells that have undergone different genetic modifications with the gene editing technique, the second sample can contain cells in which nuclear translocation of NF-κB by LPS is not inhibited at all, cells in which nuclear translocation of NF-κB is partially inhibited, and cells in which nuclear translocation of NF-κB is completely inhibited. Therefore, the second sample contains cells having unspecified morphological characteristics that differ depending on the degree of inhibition of nuclear translocation of NF-κB. That is, the second sample contains positive cells and various negative cells, and as a whole, is a sample containing a plurality of cells having unspecified morphological characteristics. A sample in which cells having specific morphological characteristics and cells having various other morphological characteristics are mixed is an example of the second sample formed of a plurality of unspecified particles in the present embodiment.

On the other hand, the first sample is created by collecting a plurality of positive cells having specific morphological characteristics. The first sample is a sample that contains positive cells having specific morphological characteristics and does not contain negative cells having other morphological characteristics. As an example, the first sample is created by subjecting cells to the same treatment and collecting cells having desired morphological characteristics in appearance. For example, by treating cells only with DMSO (Dimethyl sulfoxide), which is a solvent that does not contain LPS, positive cells that appear to have the same morphological characteristics as cells in which nuclear translocation of NF-κB by LPS is completely inhibited are obtained. Then, cells treated with DMSO are collected to create a first sample containing only cells in which nuclear translocation of NF-κB is inhibited, that is, NF-κB is localized in the cytoplasm, and which exhibits morphological characteristics similar to those of positive cells. The first sample can be created by treating a plurality of cells simultaneously. Alternatively, the first sample can be created by treating each cell individually and then mixing a plurality of cells together. Alternatively, the first sample can be created by performing some of the steps of the treatment on a plurality of cells and performing the remaining steps of the treatment all at once after mixing.

In addition, by staining the cells contained in the first sample and not staining the cells contained in the second sample, it is possible to distinguish between the cells contained in the first sample and the cells contained in the second sample. The positive cells contained in the first sample are stained by, for example, NF-κB immunostaining. Staining of the cells contained in the first sample can be performed after creating positive cells. In addition, cells can also be stained during or before creating positive cells. In FIG. 1, a positive cell is indicated by double circles. In addition, a stained positive cell contained in the first sample is indicated by double circles in a square. In addition, a negative cell is indicated by shapes other than double circles, such as a triangle in a circle, a pentagon in a circle, and a star in a circle. In addition, although the first sample and the second sample are created in separate processes, the creation of the first sample and the second sample is not limited thereto.

Then, the first sample and the second sample are mixed to create a mixed sample. The mixed sample contains cells contained in the first sample and cells contained in the second sample. The mixed sample is a test sample for classifying cells according to their morphological characteristics.

Then, a training sample necessary to create training data for machine learning is created. The training sample is created, for example, by separating a part of the mixed sample. As a result, the training sample contains cells contained in the first sample and cells contained in the second sample. It is desirable that an amount of the mixed sample is larger than an amount of the training sample. When the training sample is created by separating a part of the mixed sample, the ratio of the first sample and the second sample contained in the mixed sample and the ratio of the first sample and the second sample contained in the training sample are the same. The ratio herein is the ratio of the number of cells. In this case, the proportion of positive cells among the cells contained in the training sample is equal to the proportion of positive cells among the cells contained in the mixed sample.

In addition, the training sample and the mixed sample may be created separately. For example, by separating a part of the first sample as a training sample, separating a part of the second sample as a training sample, and mixing the remaining first sample and the remaining second sample together, it is possible to create a mixed sample. At this time, the separated part of the first sample and the separated part of the second sample may be mixed and used as a training sample, or a part of the unmixed first sample and a part of the unmixed second sample may be separately used as the training sample. Even if the training sample is created separately from the mixed sample, the ratio of the number of cells between the first sample and the second sample contained in the mixed sample is adjusted to be approximately the same as the ratio of the number of cells between the first sample and the second sample contained in the training sample. That is, the proportion of positive cells among the cells contained in the training sample is adjusted to be approximately the same as the proportion of positive cells among the cells contained in the mixed sample.

Then, waveform data indicating the morphological characteristics of cells is acquired by using the training sample, and a classification model is created that outputs identification information indicating whether or not the cell is a positive cell having specific morphological characteristics according to the waveform data. The waveform data indicating the morphological characteristics of a cell is, for example, waveform data that is obtained by using the GC method and indicates a temporal change in the intensity of light emitted from the cell. The classification model is a trained model, and is created by supervised learning using waveform data. The classification model and training processing will be described later.

Then, the cells contained in the mixed sample are classified according to their morphological characteristics by using the classification model. The classification process will be described later. By the classification, positive cells having specific morphological characteristics are classified from the mixed sample. For example, in the example of nuclear translocation of NF-κB, among the cells that have undergone gene editing treatment, cells exhibiting specific morphological characteristics in which nuclear translocation of NF-κB by LPS is inhibited (that is, NF-κB remains in the cytoplasm even after LPS stimulation) are classified as positive cells. The specific gene of the classified positive cells which is modified by gene editing is identified as a gene associated with inhibition of nuclear translocation of NF-κB by LPS.

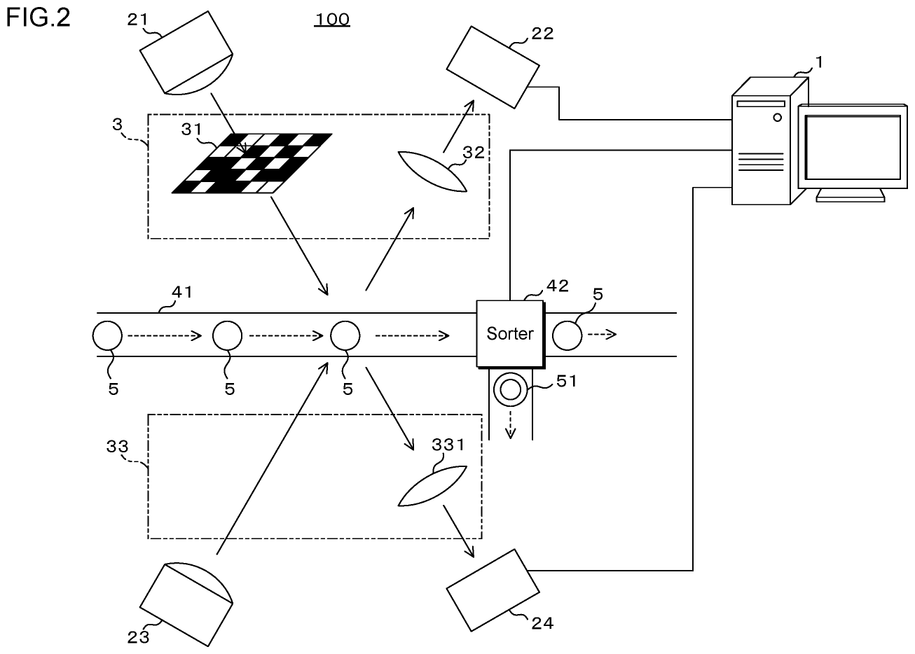

FIG. 2 is a block diagram showing a configuration example of a classification apparatus 100 according to a first embodiment for performing training and cell classification. The classification apparatus 100 includes a channel 41 through which cells flow. Cells 5 are dispersed in the fluid, and as the fluid flows through the channel 41, the individual cells 5 sequentially move through the channel 41. The classification apparatus 100 includes a light source 21 that irradiates light to the cells 5 moving through the channel 41. The light source 21 irradiates white light or monochromatic light. The light source 21 is, for example, a laser light source or an LED (light emitting diode) light source. The cell 5 irradiated with light emits light. The light emitted from the cell 5 is, for example, reflected light, scattered light, transmitted light, fluorescence, Raman scattered light, or diffracted light thereof. The classification apparatus 100 includes a detection unit 22 that detects light from the cell 5. The detection unit 22 includes a light detection sensor such as a photomultiplier tube (PMT), a line type PMT element, a photodiode, an APD (Avalanche Photo-diode), or a semiconductor optical sensor. The light detection sensor included in the detection unit 22 may be a single sensor or a multi-sensor. In FIG. 2, the path of light is indicated by solid arrows.

The classification apparatus 100 includes an optical system 3. The optical system 3 guides illumination light from the light source 21 to the cell 5 in the channel 41, and causes the light from the cell 5 to be incident on the detection unit 22. The optical system 3 includes a spatial light modulation device 31 for modulating and structuring the incident light. The classification apparatus 100 shown in FIG. 2 is configured such that the illumination light from the light source 21 is irradiated to the cell 5 through the spatial light modulation device 31. The spatial light modulation device 31 is a device for modulating light by controlling the spatial distribution (amplitude, phase, polarization, and the like) of light. The spatial light modulation device 31 has, for example, a plurality of regions on a surface on which light is incident, and the incident light is modulated differently in two or more of the plurality of regions. Here, modulation means changing the characteristics of light (any one or more of light properties such as intensity, wavelength, phase, and polarization state of light).

The spatial light modulation device 31 is, for example, a diffractive optical element (DOE), a spatial light modulator (SLM), or a digital micromirror device (DMD). In addition, when the illumination light irradiated from the light source 21 is incoherent light, the spatial light modulation device 31 is a DMD. In addition, another example of the spatial light modulation device 31 is a film or an optical filter in which a plurality of types of regions having different light transmittances are arranged randomly or in a predetermined pattern. Here, the plurality of types of regions having different light transmittances are arranged in a predetermined pattern means, for example, a state in which a plurality of types of regions having different light transmittances are arranged in a one-dimensional or two-dimensional grid pattern. In addition, a plurality of types of regions having different light transmittances are arranged randomly means that the plurality of types of regions are arranged so as to be irregularly scattered. The film or optical filter described above has at least two types of regions: a region having a first light transmittance and a region having a second transmittance different from the first light transmittance. Thus, the illumination light from the light source 21 is modulated by the spatial light modulation device 31 before being irradiated to the cell 5. For example, the illumination light from the light source 21 is converted into structured illumination light in which bright spots with different light intensities are arranged randomly or in a predetermined pattern depending on the location. Thus, the configuration in which the illumination light from the light source 21 is modulated by the spatial light modulation device 31 in the middle of the optical path from the light source 21 to irradiation to the cell 5 is also referred to as a structured illumination.

The illumination light by the structured illumination is irradiated to a specific region (illumination region) in the channel 41. When the cell 5 moves within the illumination region, the cell 5 is irradiated with the structured illumination light. Since the cell 5 moves through the region irradiated with the structured illumination light, the cell 5 is irradiated with light having characteristics such as light intensity that changes depending on the location. The cell 5 is irradiated with the structured illumination light and emits light, such as transmitted light, fluorescence, scattered light, interference light, diffracted light, or polarized light, which is emitted from the cell 5 or generated through the cell 5. Hereinafter, the light emitted from the cell 5 or generated through the cell 5 will also be referred to as light modulated by the cell 5. The light modulated by the cell 5 is detected by the detection unit 22 continuously while the cell 5 passes through the irradiation region of the channel 41. The detection unit 22 outputs an electrical signal according to the intensity of the detected light to an information processing device 1. The information processing device 1 receives waveform data obtained by converting the electrical signal into a digital signal. That is, the classification apparatus 100 can acquire waveform data indicating a temporal change in the intensity of light detected by the detection unit 22.

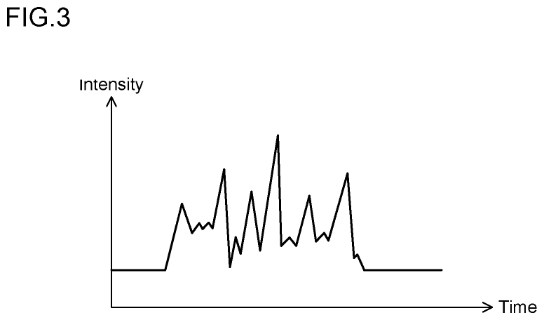

FIG. 3 is a graph showing an example of waveform data. In FIG. 3, the horizontal axis indicates time, and the vertical axis indicates the intensity of light detected by the detection unit 22. The waveform data herein is obtained by converting the signal of the light detected by the detection unit 22 into a digital signal, and is time-series data indicating a temporal change in the optical signal that reflects the morphological characteristics of the cell 5. The optical signal is a signal indicating the intensity of light detected by the detection unit 22. The waveform data is, for example, waveform data that is obtained by using the GC method and indicates a temporal change in the intensity of light emitted from the cell 5. The optical signal from the cell 5 obtained by using the GC method includes compressed morphological information of the cell. Therefore, the temporal change in the intensity of the light detected by the detection unit 22 varies according to the morphological characteristics of the cell 5, such as the size, shape, internal structure, density distribution, or color distribution. The intensity of the light from the cell 5 also changes as the intensity of the structured illumination light changes over time as the cell 5 moves within the irradiation region in the channel 41. As a result, the intensity of the light detected by the detection unit 22 changes over time, forming a waveform that changes over time as shown in FIG. 3. The waveform data indicating the temporal change in the intensity of light modulated by the cell 5, which is obtained by the structured illumination, is waveform data including compressed morphological information according to the morphological characteristics of the cell 5. For this reason, it is also possible to generate an image of the cell 5 from the waveform data obtained by the structured illumination. However, in the flow cytometer using the GC method, morphologically different cells are identified by machine learning using the waveform data as training data as it is. In addition, the classification apparatus 100 may be configured to individually acquire waveform data for a plurality of types of modulated light emitted from one cell 5.

The optical system 3 includes a lens 32 in addition to the spatial light modulation device 31. The lens 32 collects the light from the cell 5 and makes the collected light incident on the detection unit 22. In addition to the spatial light modulation device 31 and the lens 32, the optical system 3 includes optical components, such as a mirror, a lens, and a filter, in order to structure the illumination light from the light source 21, irradiate the cell 5 with the structured illumination light, and make the light from the cell 5 incident on the detection unit 22. In FIG. 2, descriptions of optical components that can be included in the optical system 3 other than the spatial light modulation device 31 and the lens 32 are omitted.

The classification apparatus 100 includes the information processing device 1. The information processing device 1 performs information processing necessary for training of a classification model and classification of cells. The detection unit 22 is connected to the information processing device 1. The detection unit 22 outputs an electrical signal according to the intensity of the detected light to the information processing device 1, and the information processing device 1 receives the electrical signal from the detection unit 22.

In addition to the light source 21, the detection unit 22, and the optical system 3, the classification apparatus 100 includes a second light source 23, a second detection unit 24, and a second optical system 33 for acquiring the intensity of light modulated by the cell 5 without going through the structuring process. The second optical system 33 includes a lens 331. Light from the second light source 23 is irradiated to the cell 5, and the light from the cell 5 is collected by the lens 331 to be incident on the second detection unit 24. In addition to the lens 331, the second optical system 33 may include optical components such as a mirror, a lens, and a filter. In FIG. 2, descriptions of optical components that can be included in the second optical system 33 other than the lens 331 are omitted.

The classification apparatus 100 determines whether or not the cell 5 is a stained cell based on optical information obtained using the second light source 23, the second detection unit 24, and the second optical system 33. In the classification apparatus 100 shown in FIG. 2, the second light source 23 irradiates the cell 5 with illumination light that is not structured, and the second detection unit 24 detects the light modulated by the cell 5. When the cell 5 is a fluorescently stained cell, the second detection unit 24 detects the fluorescence emitted from the cell 5 and outputs information regarding the detected fluorescence intensity to the information processing device 1. The information processing device 1 determines whether or not the cell 5 is a stained cell based on the information regarding the fluorescence intensity from the second detection unit 24. That is, the classification apparatus 100 acquires optical information for determining whether or not the cell 5 is a stained cell by using the second light source 23, the second detection unit 24, and the second optical system 33 for acquiring the intensity of light emitted from the cell 5 without going through the structuring process. The information processing device 1 determines whether or not the cell 5 is a stained cell based on the acquired optical information. Although FIG. 2 shows a configuration in which the second optical system 33 for determining whether or not the cell 5 is a stained cell does not include a spatial light modulation device, the classification apparatus 100 may be configured to emit the structured illumination light to the cell 5 and determine whether or not the cell 5 is a stained cell.

A sorter 42 may be further connected to the channel 41. The sorter 42 sorts specific cells from the cells 5 that have moved through the channel 41. For example, when the cells 5 that have moved through the channel 41 are the specific cells 51, the sorter 42 sorts the cells 51 by changing the movement path. The sorter 42 is connected to the information processing device 1 and is controlled by the information processing device 1. The sorter 42 sorts cells under the control of the information processing device 1. The cells 51 to be sorted are positive cells contained in the second sample. The information processing device 1 classifies positive cells and negative cells based on the created classification model, and the sorter 42 sorts the positive cells contained in the second sample. In the above-described example of NF-κB, the sorter 42 sorts cells in which nuclear translocation of NF-κB by LPS is inhibited, that is, cells exhibiting specific morphological characteristics in which NF-κB remains in the cytoplasm, among the cells 5 that have moved through the channel 41, as positive cells.

In addition, the sorter 42 separates cells that are stained (stained cells) and cells that are not stained (non-stained cells) from each other under the control of the information processing device 1. The information processing device 1 classifies stained cells and non-stained cells based on the acquired information, and the sorter 42 sorts the non-stained cells. That is, the information processing device 1 can simultaneously perform classification of positive cells and negative cells based on the created classification model and classification of cells based on the presence or absence of staining. The sorter 42 identifies and sorts non-stained positive cells from the cells contained in the mixed sample under the control of the information processing device 1. That is, positive cells contained in the second sample are sorted. For example, in the above-described example of NF-κB, only cells in which nuclear translocation of NF-κB by LPS is inhibited by gene editing are sorted. In FIG. 2, the path of cells is indicated by dashed arrows.

Although FIG. 2 describes a case where the sorter 42 sorts non-stained positive cells based on both classification into positive cells and negative cells and classification of cells based on the presence or absence of staining, the present disclosure is not limited thereto. For example, the classification apparatus 100 can have a configuration in which a sorter that sorts positive cells by classification into positive cells and negative cells and a sorter that sorts non-stained positive cells by classification of sorted positive cells into stained cells and non-stained cells are separately arranged.

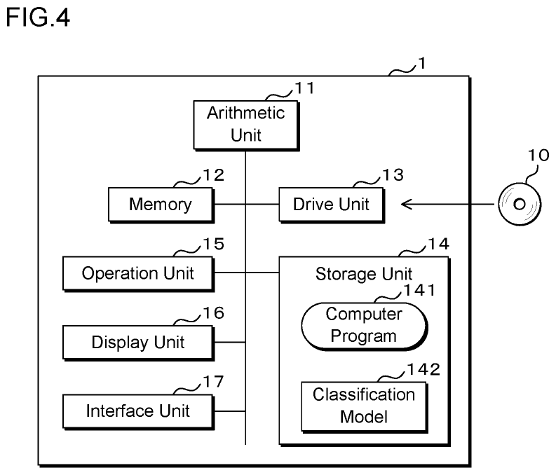

FIG. 4 is a block diagram showing an example of the internal configuration of the information processing device 1. The information processing device 1 is, for example, a computer such as a personal computer or a server device. The information processing device 1 includes an arithmetic unit 11, a memory 12, a drive unit 13, a storage unit 14, an operation unit 15, a display unit 16, and an interface unit 17. The arithmetic unit 11 is configured by using, for example, a CPU (Central Processing Unit), a GPU (Graphics Processing Unit), or a multi-core CPU. The arithmetic unit 11 may be configured by using a quantum computer. The memory 12 stores temporary data generated along with calculations. The memory 12 is, for example, a RAM (Random Access Memory). The drive unit 13 reads information from a recording medium 10 such as an optical disc or a portable memory.

The storage unit 14 is nonvolatile, and is, for example, a hard disk or a nonvolatile semiconductor memory. The operation unit 15 receives an input of information, such as text, by receiving an operation from the user. The operation unit 15 is, for example, a touch panel, a keyboard, or a pointing device. The display unit 16 displays an image. The display unit 16 is, for example, a liquid crystal display or an EL display (Electroluminescent Display). The operation unit 15 and the display unit 16 may be integrated. The interface unit 17 is connected to the detection unit 22 and the sorter 42. The interface unit 17 transmits and receives signals to and from the detection unit 22 and the sorter 42.

The arithmetic unit 11 causes the drive unit 13 to read a computer program 141 recorded on the recording medium 10, and stores the read computer program 141 in the storage unit 14. The arithmetic unit 11 performs processing necessary for the information processing device 1 according to the computer program 141. The computer program 141 may be downloaded from the outside of the information processing device 1. Alternatively, the computer program 141 may be stored in the storage unit 14 in advance. In these cases, the information processing device 1 may not include the drive unit 13. The information processing device 1 may be configured by a plurality of computers.

The information processing device 1 includes a classification model 142 used to determine whether or not the cell 5 is a positive cell based on waveform data. The classification model 142 is a trained model that has been trained to output identification information indicating whether or not the cell 5 is a positive cell when waveform data is input. The information processing device 1 performs processing for training the classification model 142 and processing for classifying the cells 5 using the classification model 142. The classification model 142 is realized by the arithmetic unit 11 executing information processing according to the computer program 141. The storage unit 14 stores data necessary for realizing the classification model 142. The classification model 142 may be configured by using hardware. For example, the classification model 142 may be configured by hardware including a processor and a memory that stores necessary programs and data. The classification model 142 may be realized by using a quantum computer. Alternatively, the classification model 142 may be provided outside the information processing device 1, and the information processing device 1 may perform processing by using the external classification model 142. For example, the classification model 142 may be configured on the cloud.

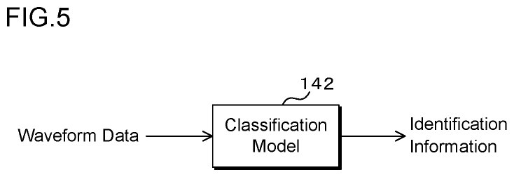

FIG. 5 is a conceptual diagram showing the functions of the classification model 142. Waveform data obtained from each cell 5 is input to the classification model 142. The classification model 142 is trained to output identification information indicating whether or not the cell 5 is a positive cell having specific morphological characteristics when waveform data is input. For example, the classification model 142 is configured by a neural network or a support vector machine.

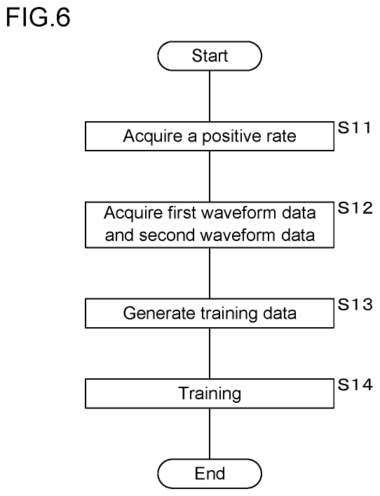

The information processing device 1 executes a classification model generation method by performing processing for training the classification model 142. FIG. 6 is a flowchart showing an example of the procedure of processing for training the classification model 142. Hereinafter, the step is abbreviated as S. The arithmetic unit 11 performs the following processing according to the computer program 141. The information processing device 1 acquires a positive rate, which is the proportion of positive cells having specific morphological characteristics among all the cells contained in the mixed sample (S11). The positive rate is the proportion of positive cells among the cells contained in the mixed sample. As described above, a training sample and a mixed sample are prepared so that the positive rates are approximately equal. For example, when using a part of the mixed sample as a training sample, the positive rate can be obtained by measuring a part of the training sample. In this case, for example, the positive rate can be obtained by observing each cell contained in the training sample using an observation means, such as a microscope, and measuring the number of positive cells and the number of negative cells or measuring the ratio between positive cells and negative cells. In S11, the user inputs the positive rate by operating the operation unit 15, so that the information processing device 1 acquires the positive rate. The arithmetic unit 11 stores the acquired positive rate in the storage unit 14.

Alternatively, the positive rate can be obtained by calculation. For example, the positive rate can be calculated based on the number of cells (positive cells) contained in the first sample, the number of cells contained in the second sample, and the number of positive cells contained in the second sample. Alternatively, when the second sample contains cells having various morphological characteristics and the number of positive cells contained in the second sample is very small, the ratio of positive cells contained in the first sample to all the cells contained in the first sample and the second sample is slightly smaller than the ratio of positive cells to all the cells contained in the first sample and the second sample, but these ratios may be approximately the same value. In such a case, the ratio of the number of positive cells contained in the first sample to the total number of cells contained in the first sample and the second sample can be used as the positive rate. That is, from the number of cells contained in each of the first sample and the second sample or the ratio between the cells contained in the first sample and the cells contained in the second sample, the ratio of the number of positive cells contained in the first sample to the total number of cells is calculated as the positive rate. In S11, the user inputs the calculated positive rate by operating the operation unit 15, so that the information processing device 1 acquires the positive rate. Alternatively, the user may input the number of cells contained in each of the first sample and the second sample or the ratio between the cells contained in the first sample and the cells contained in the second sample by operating the operation unit 15, and the arithmetic unit 11 may acquire the positive rate by calculating the positive rate based on the input value. The arithmetic unit 11 stores the acquired positive rate in the storage unit 14.

Then, the information processing device 1 acquires first waveform data obtained from the cells contained in the first sample and second waveform data obtained from the cells contained in the second sample (S12). Each cell 5 contained in the training sample flows through the channel 41, and illumination light by the structured illumination is irradiated to the cell 5 using the light source 21 and the spatial light modulation device 31. Due to the irradiation of the structured illumination light, the cell 5 emits modulated light (light modulated by the cell 5), such as scattered light, and the emitted modulated light is detected by the detection unit 22. The detection unit 22 outputs a signal according to the intensity of the detected light to the information processing device 1, and the information processing device 1 receives the signal from the detection unit 22 by using the interface unit 17. The arithmetic unit 11 generates waveform data indicating a temporal change in the intensity of the light detected by the detection unit 22 based on the optical signal from the detection unit 22.

When using a part of the mixed sample as a training sample, the training sample contains a mixture of cells contained in the first sample and cells contained in the second sample. The classification apparatus 100 detects staining of each cell and determines whether the cell is a cell from the first sample or a cell from the second sample. As described above, the classification apparatus 100 has a function for acquiring the optical signal from the stained cell 5 (for example, fluorescence from the fluorescently stained cell), in addition to the function for acquiring waveform data using the GC method. That is, the second light source 23 emits unstructured illumination light to the cell 5, and the second detection unit 24 detects the fluorescence emitted from the fluorescently stained cell 5 contained in the first sample. The arithmetic unit 11 determines whether or not the cell 5 is stained based on the signal from the second detection unit 24. Alternatively, in the classification apparatus 100, the second detection unit 24 detects light that has passed through a color filter according to the staining agent, and the arithmetic unit 11 determines whether or not the cell 5 is stained based on the signal from the second detection unit 24. When the cell 5 is stained, the arithmetic unit 11 sets the waveform data acquired by the GC method as the first waveform data. When the cell 5 is not stained, the arithmetic unit 11 sets the waveform data acquired by the GC method as the second waveform data.

When a part of the first sample and a part of the second sample are present as a training sample without being mixed, each cell 5 contained in the first sample in the training sample passes through the channel 41, illumination light by the structured illumination is irradiated to the cell 5, and the information processing device 1 acquires the first waveform data. In addition, each cell 5 contained in the second sample in the training sample flows through the channel 41, illumination light by the structured illumination is irradiated to the cell 5, and the information processing device 1 acquires the second waveform data.

The first waveform data obtained in S12 indicates the morphological characteristics of positive cells. The second waveform data is waveform data acquired from unspecified cells with different morphological characteristics contained in the second sample, and accordingly, indicates waveforms with various shapes. Although the second waveform data indicates the morphological characteristics of each cell, it is not clear whether the cell that generates the second waveform data is a positive cell or a negative cell. In S12, for each of the plurality of cells contained in the training sample, waveform data is acquired as the first waveform data or the second waveform data. The arithmetic unit 11 stores the first waveform data and the second waveform data in the storage unit 14. The processing of S12 corresponds to a data acquisition unit. Although an example in which S11 is executed before S12 is described in the flowchart shown in FIG. 6, the order in which S11 and S12 are executed may be reversed.

Then, the information processing device 1 generates training data for training (S13). The training data includes a positive rate, a plurality of pieces of first waveform data, information indicating that the first waveform data is obtained from cells contained in the first sample, a plurality of pieces of second waveform data, and information indicating that the second waveform data is obtained from cells contained in the second sample. The information indicating that the first waveform data is obtained from cells contained in the first sample is associated with each piece of first waveform data. The information indicating that the second waveform data is obtained from cells contained in the second sample is associated with each piece of second waveform data.

As the information indicating that the first waveform data is obtained from cells contained in the first sample, information indicating positive cells may be associated with the first waveform data. As the information indicating that the second waveform data is obtained from cells contained in the second sample, information indicating that the cells are unspecified cells having different morphological characteristics or information regarding the test substance in contact with the cell or gene editing may be associated with the second waveform data. Alternatively, the information indicating that the second waveform data is obtained from cells contained in the second sample may be expressed by not associating information regarding the cells with the second waveform data. The arithmetic unit 11 stores the training data in the storage unit 14.

Then, the information processing device 1 trains the classification model 142 (S14). In S14, the arithmetic unit 11 performs training using a PU (Positive and Unlabeled Learning) classification method. In S14, the arithmetic unit 11 inputs the first waveform data or the second waveform data to the classification model 142. The classification model 142 outputs identification information indicating whether or not the cell that has generated the waveform data is a positive cell. The arithmetic unit 11 adjusts the calculation parameters of the classification model 142 so that appropriate identification information is output according to the waveform data.

In S14, the arithmetic unit 11 sequentially inputs each of the first waveform data and the second waveform data to the classification model 142. The arithmetic unit 11 performs two-class classification (PU classification) based on the first waveform data obtained from the first sample containing only positive cells and the second waveform data obtained from the second sample containing both positive cells and non-positive cells. In the PU classification, an objective function is set by using positive data, unlabeled data, and the proportion of positive cases in the data set, and the classification model 142 that minimizes the objective function is created. As a loss function, in addition to the commonly used 0/1 loss function, a surrogate loss function that is easy to optimize can be used. As the surrogate loss function, both convex and non-convex functions can be used. For example, a logistic loss function, a square loss function, or a two-stage hinge loss function can be used as the surrogate loss function. A classification model based on PU classification can be created using the equations described in Proceedings of Machine Learning Research 37:1386 to 1394, 2015, for example.

The arithmetic unit 11 performs machine learning of the classification model 142 by repeating the processing for adjusting the calculation parameters of the classification model 142 using the training data. When the classification model 142 is a neural network, the adjustment of the calculation parameters of each node is repeated. The classification model 142 is trained to output identification information indicating that the cell is a positive cell when waveform data obtained from the positive cell is input and to output identification information indicating that the cell is not a positive cell when waveform data obtained from the negative cell is input. The arithmetic unit 11 stores learned data, in which the adjusted final parameters are recorded, in the storage unit 14. In this manner, the trained classification model 142 is generated. The processing of S14 corresponds to a classification model generation unit. After S14 ends, the information processing device 1 ends the process for training the classification model 142.

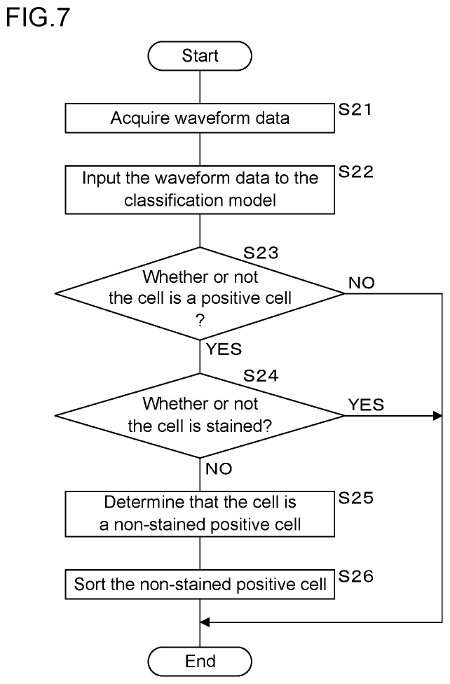

The classification apparatus 100 classifies cells using the trained classification model 142. By classifying cells using the trained classification model 142, the particle classification method is executed. FIG. 7 is a flowchart showing an example of the procedure of processing performed by the information processing device 1 in order to classify cells. One cell 5 contained in the mixed sample moves through the channel 41. By using the light source 21 and the spatial light modulation device 31, illumination light by the structured illumination is irradiated to the cell 5. The cell 5 emits light, such as fluorescence, and the emitted light is detected by the detection unit 22. The detection unit 22 outputs an electrical signal to the information processing device 1 according to the intensity of the detected light. The electrical signal output from the detection unit 22 is received by the interface unit 17 of the information processing device 1 as waveform data through a DAQ (Data acquisition) device (not shown in FIG. 2) that converts the electrical signal into a digital signal. The information processing device 1 acquires waveform data caused by the cell 5 (S21). In S21, the arithmetic unit 11 acquires waveform data that is generated based on the electrical signal from the detection unit 22 and indicates a temporal change in the intensity of the light detected by the detection unit 22.

The information processing device 1 inputs the acquired waveform data to the classification model 142 (S22). In S22, the arithmetic unit 11 inputs the waveform data to the classification model 142, and causes the classification model 142 to perform processing. At this time, the arithmetic unit 11 does not input the positive rate to the classification model 142. In response to the input of the waveform data, the classification model 142 performs processing for outputting identification information indicating whether or not the cell 5 is a positive cell having specific morphological characteristics. The processing of S22 corresponds to a data input unit. The information processing device 1 determines whether or not the cell 5 is a positive cell based on the identification information output from the classification model 142 (S23). In S23, the arithmetic unit 11 determines that the cell 5 is a positive cell when the identification information indicates that the cell 5 is a positive cell, and determines that the cell 5 is not a positive cell when the identification information indicates that the cell 5 is not a positive cell. When the cell 5 is not a positive cell (S23: NO), the information processing device 1 ends the process for classifying cells.

When the cell 5 is a positive cell (S23: YES), the information processing device 1 then determines whether or not the cell 5 determined to be a positive cell in S23 is stained (S24). In S24, the arithmetic unit 11 determines whether or not the cell 5 is stained based on the detection result of the second detection unit 24. For example, the arithmetic unit 11 makes the determination based on the intensity of light having a specific wavelength included in the detection result. When the cell 5 is stained (S24: YES), the information processing device 1 ends the process for classifying cells.

The stained cell is a cell contained in the first sample. In the above-described example, the stained cell is a cell treated with DMSO that does not contain LPS, and is a cell in which nuclear translocation of NF-κB has not occurred. That is, in the above-described example, the cell contained in the first sample is a positive cell having specific morphological characteristics. However, the cell contained in the first sample appears to be a positive cell after being artificially treated to have the specific morphological characteristics. Thus, the positive cell contained in the first sample exhibits specific morphological characteristics in which NF-κB remains in the cytoplasm without nuclear translocation, but is not necessarily a cell in which nuclear translocation of NF-κB is inhibited by test substance treatment or gene editing.

When the cell 5 determined to be a positive cell is not stained (S24: NO), the information processing device 1 determines that the cell 5 is a non-stained positive cell (cell 51) (S25). The processing of S25 corresponds to a determination unit. Then, the information processing device 1 sorts the non-stained positive cell by using the sorter 42 (S26). In S26, the arithmetic unit 11 transmits a control signal from the interface unit 17 to the sorter 42 to cause the sorter 42 to sort the cell 51. The sorter 42 sorts the cell 51 according to the control signal. There are various methods for sorting the cell 51 by the sorter 42. For example, when the cell 51 determined to be a non-stained positive cell has flowed to the sorter 42, the sorter 42 sorts the cell 51 by applying an electric charge to the droplet containing the cell 51 and applying a voltage to the droplet so that the movement path of the droplet containing the cell 51 is changed. Alternatively, the sorter 42 can also sort the cell 51 by generating a pulse flow when the cell 51 has flowed to the sorter 42 so that the movement path of the cell 51 is changed.

The sorted cell 51 is a non-stained positive cell. Since the non-stained cell is a cell contained in the second sample, the non-stained positive cell is a positive cell among the cells contained in the second sample. For example, in the above-described example, this cell is a cell in which nuclear translocation of NF-κB by LPS is inhibited by genetic modification. For example, the nuclear translocation of NF-κB is inhibited by modifying a gene related to nuclear translocation of NF-κB by LPS. By sorting the non-stained positive cell, cells in which a gene related to nuclear translocation of NF-κB by LPS has been modified are sorted. The sorted cell 51 can be stored as necessary, and can be provided for tests to analyze changes occurring in the cells (for example, changes in gene products or genetically modified sites).

After S26 ends, the information processing device 1 ends the process for classifying cells. A plurality of cells 5 contained in the mixed sample sequentially flow through the channel 41, and the processing of S21 to S26 is performed each time each cell 5 moves through the channel 41. In this manner, the classification apparatus 100 classifies the cells contained in the mixed sample. Among the cells contained in the mixed sample, non-stained positive cells are sorted. For example, in the above-described example, non-stained positive cells are cells in which a phenomenon that nuclear translocation of NF-κB by LPS stimulation is inhibited by genetic modification has occurred.

As described in detail above, in the present embodiment, the classification model 142 is trained by using the training data including the first waveform data obtained from cells contained in the first sample formed of positive cells, the second waveform data obtained from cells contained in the second sample formed of unspecified cells, and the positive rate in the mixed sample. Positive cells are cells exhibiting specific morphological characteristics, while other negative cells include unspecified cells having different morphological characteristics. Although the waveform data of negative cells cannot be used as training data, it is possible to generate the classification model 142 by using PU classification using the second waveform data obtained from unspecified cells as training data. The classification model 142 outputs identification information indicating whether or not the cell related to the waveform data is a positive cell when waveform data is input. Even if the waveform data of negative cells cannot be used as training data, the classification model 142 that outputs identification information can be generated. In addition, identification of cells using the classification model 142 becomes possible by a flow cytometer that uses the GC method.

In the present embodiment, a part of the mixed sample obtained by mixing the first sample and the second sample is used as a training sample, and particles contained in the remaining mixed sample are classified by using the classification model 142. Training data for generating the classification model 142 is obtained using the training sample. The training sample used for training and the mixed sample, which is the target of particle classification, are essentially the same sample. Therefore, cell classification is performed accurately.

According to the present embodiment, cells having specific morphological characteristics, among a plurality of cells having various morphological characteristics, can be accurately and quickly identified. For example, when various genes are modified by gene editing and there is a change in the nuclear translocation of NF-κB due to LPS stimulation, cells in which nuclear translocation of NF-κB due to LPS stimulation is inhibited, among a plurality of cells having different degrees of nuclear translocation of NF-κB, can be identified and sorted. By examining the genes contained in the sorted cells, it is possible to identify genes involved in the nuclear translocation of NF-κB by LPS. In this manner, it is possible to cause cells to express specific morphological characteristics and identify genes involved in changes in phenotype of cells.

### Second Embodiment

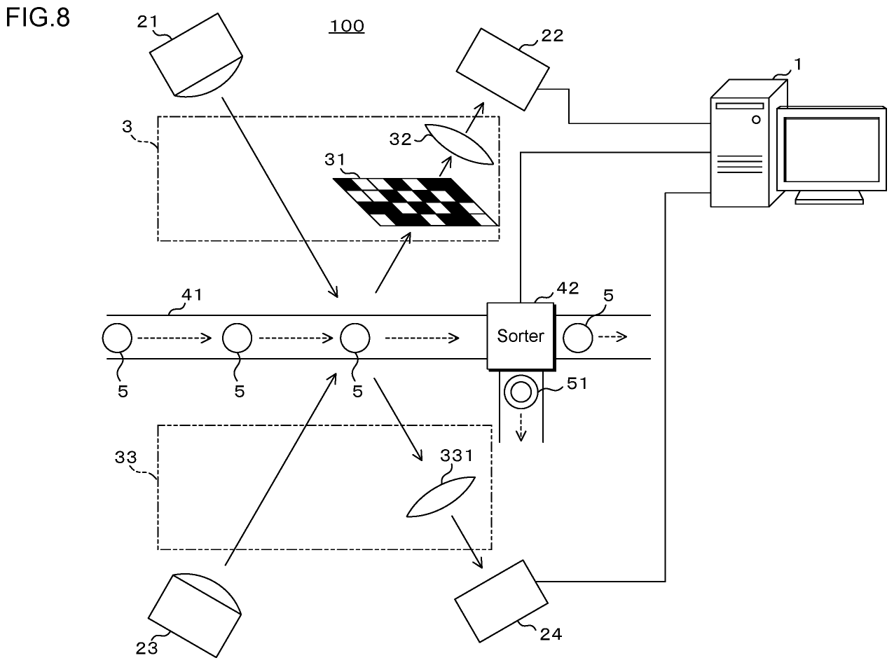

FIG. 8 is a block diagram showing a configuration example of a classification apparatus 100 according to a second embodiment. In the second embodiment, the configuration of the optical system 3 is different from that in the first embodiment shown in FIG. 2. The configuration of components other than the optical system 3 is the same as that in the first embodiment. Unlike in the first embodiment, the illumination light from the light source 21 is irradiated to the cell 5 without passing through the spatial light modulation device 31. On the other hand, the light from the cell 5 passes through the spatial light modulation device 31 and is collected by the lens 32 to be incident on the detection unit 22. The modulated light from the cell 5 becomes structured modulated light by passing through the spatial light modulation device 31 and the light is detected by the detection unit 22. The configuration in which the light from the cell 5 is modulated by the spatial light modulation device 31 in the middle of the optical path from the cell 5 to the detection unit 22 as described above is also referred to as structured detection. For example, the intensity of the modulated light from the cell 5 detected by the detection unit 22 changes over time due to the spatial light modulation device 31. The waveform data indicating a temporal change in the intensity of light from the cell 5 that is detected by the detection unit 22 by structured detection includes compressed morphological information of the cell 5, as in the case of the structured illumination in the first embodiment. The waveform of the waveform data changes according to the morphological characteristics of the cell 5.

In the second embodiment as well, the classification apparatus 100 can acquire waveform data indicating a temporal change in the intensity of light detected by the detection unit 22. The waveform data indicates a temporal change in the intensity of light emitted from the cell 5. As in the first embodiment, the waveform data indicates the morphological characteristics of the cell 5. The optical system 3 includes optical components, such as a mirror, a lens, and a filter, in addition to the spatial light modulation device 31 and the lens 32. In FIG. 8, descriptions of optical components included in the optical system 3 other than the spatial light modulation device 31 and the lens 32 are omitted. In the structured detection, optical components used in the structured illumination in the first embodiment can be similarly used as the spatial light modulation device 31. In the classification apparatus 100 according to the second embodiment, for example, a film or an optical filter in which a plurality of types of regions having different light transmittances are arranged randomly or in a predetermined pattern can be used as the spatial light modulation device 31. FIG. 8 shows an example of a film in which two types of regions having different light transmittances are arranged in a two-dimensional grid pattern.

In the second embodiment as well, the information processing device 1 generates the classification model 142 by performing the processing of S11 to S14 as in the first embodiment. In addition, as in the first embodiment, the information processing device 1 determines whether or not the cell is a positive cell having specific morphological characteristics and sorts non-stained positive cells, among a plurality of cells having various morphological characteristics, by performing the processing of S21 to S26. In the second embodiment as well, the classification model 142 can be generated even if waveform data of negative cells cannot be used as training data. Therefore, identification of cells using the classification model 142 becomes possible.

In the first and second embodiments described above, an illustrative embodiment in which the classification apparatus 100 includes the sorter 42 to sort cells is shown. However, the classification apparatus 100 may not include the sorter 42. In this illustrative embodiment, the information processing device 1 omits the processing of S26. The classification model generation method and the particle classification method can also be used in an analyzer that does not have a function of sorting the identified cells. In the first and second embodiments, an illustrative embodiment in which the cells contained in the first sample are stained is shown. However, in the classification model generation method and the particle classification method, it is also possible to distinguish between the cells by using other methods in which the cells contained in the first sample are not stained.

In the first and second embodiments, an illustrative embodiment is shown in which the first sample and the second sample are created from the same sample, a part of the first sample and a part of the second sample are used as training samples, and cells contained in a mixed sample made by mixing the remaining first and second samples together are classified. In the particle classification method, however, waveform data may be acquired from the cells contained in each of the first sample and the second sample other than the training sample, without creating a mixed sample, to classify the cells. In the classification model generation method and the particle classification method, the training sample and the mixed sample may be created from different samples. For example, when the first sample and the second sample can be generated with good reproducibility, the classification model 142 may be generated by using the first sample and the second sample as training samples in the classification model generation method, and cells contained in a mixed sample prepared by using newly and separately created first and second samples may be classified in the particle classification method. The positive rate in the mixed sample to be analyzed is preferably a value close to, and more preferably the same value as, the positive rate in all the cells contained in the first sample and the second sample that are training samples.

In the first and second embodiments, an illustrative embodiment in which the classification model generation method and the particle classification method are executed using the same information processing device 1 is shown. However, the classification model generation method and the particle classification method may be executed using different information processing devices. For example, the classification model generation method and the particle classification method may be executed by different classification apparatuses. For example, the classification apparatus 100 may include an information processing device for executing the classification model generation method and an information processing device for executing the particle classification method. For example, the information processing device for executing the particle classification method includes the classification model 142 by storing learned data recording the parameters of the classification model 142 trained by the classification model generation method.

The information processing device for executing the classification model generation method and the information processing device for executing the particle classification method may have different configurations. For example, in the information processing device for executing the particle classification method, the classification model 142 may be implemented using an FPGA (Field Programmable Gate Array). The circuit of the FPGA is configured based on the parameters of the classification model 142 trained by the classification model generation method, and the FPGA executes the processing of the classification model 142. When sorting cells using the sorter 42, it is necessary to perform the processing in real time. When the classification model 142 is implemented using an FPGA, it is easier to speed up the processing of the classification model 142 than in a form in which the classification model 142 is implemented using a computer program. Therefore, in the form in which the classification model 142 is implemented using an FPGA, it is possible to easily perform the processing for sorting cells by using the sorter 42.

In the first and second embodiments, as a classification model generation method and a particle classification method, an example of identifying cells exhibiting a phenotypic change in which nuclear translocation of NF-κB by LPS is inhibited and accordingly NF-κB does not translocate to the nucleus even if LPS stimulation is applied, among cells whose genes have been variously modified by gene editing, is shown. However, the use of the classification model generation method and particle classification method is not limited to this. In addition to the examples in the first and second embodiments, in order to identify cells whose phenotype has changed to those with specific morphological characteristics among the cells whose genes have been variously modified by gene editing, the classification model generation method and the particle classification method can be used. In addition to these, in the classification model generation method and the particle classification method, cells are brought into contact with various test substances so that a cell with a phenotype having specific morphological characteristics caused by the contact can be identified. Therefore, it is possible to perform an evaluation to select, from a number of test substances, a test substance that changes a cell to a phenotype having specific morphological characteristics of interest. In addition, the classification model generation method and the particle classification method can be used for evaluation to bring cells into contact with various test substances and select, among a number of test substances, a test substance that inhibits the effect of a certain agent (a specific chemical, physiologically active substance, or the like) that causes cells having specific morphological characteristics to change their phenotype. In addition, in the classification model generation method and the particle classification method, cells can be treated using methods other than gene introduction and contact with test substances, such as heat treatment and radiation emission, and among the methods, a method for making the cells express specific morphological characteristics can be selected.

In the first and second embodiments, an example is shown in which particles are cells. However, in the classification model generation method and the particle classification method, particles other than cells may be used. Particles are preferably biological particles, but are not limited thereto. For example, particles targeted in the classification model generation method and the particle classification method may be microorganisms such as bacteria, yeast, and plankton, tissues within organisms, organs within organisms, or fine particles such as beads, pollen, and particulate matter.

The present invention is not limited to the content of the above-described embodiments, and various changes can be made within the scope of the claims. That is, embodiments obtained by combining technical means appropriately changed within the scope of the claims are also included in the technical scope of the present invention.

### Note 1

An information processing device, comprising:
a processor; and
a memory, wherein the processor is operable to:
acquire first waveform data, which is obtained by irradiating light to particles contained in a first sample formed of particles having specific morphological characteristics and indicates morphological characteristics of the particles, and second waveform data indicating morphological characteristics of particles contained in a second sample formed of a plurality of unspecified particles; and
generate a classification model, which outputs identification information indicating whether or not a particle has the specific morphological characteristics when waveform data indicating morphological characteristics of the particle is input, by training using training data including the first waveform data, information indicating that the first waveform data has been obtained from particles contained in the first sample, the second waveform data, information indicating that the second waveform data has been obtained from particles contained in the second sample, and a positive rate that is a proportion of particles having the specific morphological characteristics among all particles contained in the first sample and the second sample.

### Note 2

A non-transitory recording medium recording a computer program causing a computer to execute processing of:
inputting waveform data indicating morphological characteristics of a particle to a classification model that outputs identification information indicating whether or not the particle has specific morphological characteristics when waveform data, which is obtained by irradiating light to the particle and indicates morphological characteristics of the particle, is input; and
determining whether or not the particle has the specific morphological characteristics based on the identification information output from the classification model,
wherein the classification model is trained by using training data including first waveform data indicating morphological characteristics of particles contained in a first sample formed of particles having the specific morphological characteristics, information indicating that the first waveform data has been obtained from particles contained in the first sample, second waveform data indicating morphological characteristics of particles contained in a second sample formed of a plurality of unspecified particles, information indicating that the second waveform data has been obtained from particles contained in the second sample, and a positive rate that is a proportion of particles having the specific morphological characteristics among all particles contained in the first sample and the second sample.

### Note 3

An information processing device, comprising:
a processor; and
a memory, wherein the processor is operable to:
input waveform data indicating morphological characteristics of a particle to a classification model that outputs identification information indicating whether or not the particle has specific morphological characteristics when waveform data, which is obtained by irradiating light to the particle and indicates morphological characteristics of the particle, is input; and
determine whether or not the particle has the specific morphological characteristics based on the identification information output from the classification model,
wherein the classification model is trained by using training data including first waveform data indicating morphological characteristics of particles contained in a first sample formed of particles having the specific morphological characteristics, information indicating that the first waveform data has been obtained from particles contained in the first sample, second waveform data indicating morphological characteristics of particles contained in a second sample formed of a plurality of unspecified particles, information indicating that the second waveform data has been obtained from particles contained in the second sample, and a positive rate that is a proportion of particles having the specific morphological characteristics among all particles contained in the first sample and the second sample.

It is to be noted that, as used herein and in the appended claims, the singular forms “a”, “an”, and “the” include plural referents unless the context clearly dictates otherwise. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

It is to be noted that the disclosed embodiment is illustrative and not restrictive in all aspects. The scope of the present invention is defined by the appended claims rather than by the description preceding them, and all changes that fall within metes and bounds of the claims, or equivalence of such metes and bounds thereof are therefore intended to be embraced by the claims.

1-11. (canceled)

12. A classification model generation method, comprising:
acquiring first waveform data, which is obtained by irradiating light to particles contained in a first sample formed of particles having specific morphological characteristics and indicates morphological characteristics of the particles, and second waveform data indicating morphological characteristics of particles contained in a second sample formed of a plurality of unspecified particles; and
generating a classification model, which outputs identification information indicating whether or not a particle has the specific morphological characteristics when waveform data indicating morphological characteristics of the particle is input, by training using training data including the first waveform data, information indicating that the first waveform data has been obtained from particles contained in the first sample, the second waveform data, information indicating that the second waveform data has been obtained from particles contained in the second sample, and a positive rate that is a proportion of particles having the specific morphological characteristics among all particles contained in the first sample and the second sample.

13. The classification model generation method according to claim 12,
wherein the positive rate is a value obtained by measuring a proportion of particles having the specific morphological characteristics contained in a mixed sample obtained by mixing the first sample and the second sample together or a value obtained by calculating a proportion of particles having the specific morphological characteristics among all particles contained in the first sample and the second sample.

14. The classification model generation method according to claim 12,
wherein the waveform data is waveform data indicating a temporal change in an intensity of light emitted from a particle irradiated with light by a structured illumination or waveform data indicating a temporal change in an intensity of light detected by structuring light from a particle irradiated with light.

15. The classification model generation method according to claim 12,
wherein, by using a part of a mixed sample obtained by mixing the first sample and the second sample together as a training sample, the first waveform data and the second waveform data obtained from particles contained in the training sample are acquired as waveform data contained in the training data, and
the classification model is trained to output identification information indicating whether or not a particle contained in the mixed sample has the specific morphological characteristics when waveform data obtained from the particle is input.

16. A particle classification method, comprising:
inputting waveform data indicating morphological characteristics of a particle to a classification model that outputs identification information indicating whether or not the particle has specific morphological characteristics when waveform data, which is obtained by irradiating light to the particle and indicates morphological characteristics of the particle, is input; and
determining whether or not the particle has the specific morphological characteristics based on the identification information output from the classification model,
wherein the classification model is trained by using training data including first waveform data indicating morphological characteristics of particles contained in a first sample formed of particles having the specific morphological characteristics, information indicating that the first waveform data has been obtained from particles contained in the first sample, second waveform data indicating morphological characteristics of particles contained in a second sample formed of a plurality of unspecified particles, information indicating that the second waveform data has been obtained from particles contained in the second sample, and a positive rate that is a proportion of particles having the specific morphological characteristics among all particles contained in the first sample and the second sample.

17. The particle classification method according to claim 16,
wherein waveform data indicating morphological characteristics of particles contained in a mixed sample obtained by mixing the first sample and the second sample together is acquired,
waveform data obtained from the particles contained in the mixed sample is input to the classification model, and
whether or not each particle contained in the mixed sample has the specific morphological characteristics is determined based on identification information output from the classification model.

18. The particle classification method according to claim 17,
wherein particles contained in the first sample are stained,
particles contained in the second sample are not stained, and
particles that are not stained and have the specific morphological characteristics are identified based on presence or absence of staining of each particle determined to be a particle having the specific morphological characteristics.

19. A non-transitory recording medium recording a computer program causing a computer to execute processing of:
acquiring first waveform data, which is obtained by irradiating light to particles contained in a first sample formed of particles having specific morphological characteristics and indicates morphological characteristics of the particles, and second waveform data indicating morphological characteristics of particles contained in a second sample formed of a plurality of unspecified particles; and
generating a classification model, which outputs identification information indicating whether or not a particle has the specific morphological characteristics when waveform data indicating morphological characteristics of the particle is input, by training using training data including the first waveform data, information indicating that the first waveform data has been obtained from particles contained in the first sample, the second waveform data, information indicating that the second waveform data has been obtained from particles contained in the second sample, and a positive rate that is a proportion of particles having the specific morphological characteristics among all particles contained in the first sample and the second sample.
